(19) United States
(12) Patent Application Publication
(10) Pub. No.: US 2007/0142626 A1
(43) Pub. Date: Jun. 21, 2007

(54) CROSS-LINKING REAGENTS FOR HEMOGLOBIN AND HEMOGLOBIN PRODUCTS CROSS-LINKED THEREWITH

(76) Inventors: Ronald Kluger, Toronto (CA); Dongxin Hu, Toronto (CA)

Correspondence Address: BURNS & LEVINSON, LLP, 125 SUMMER STREET, BOSTON, MA, 02110, US

(21) Appl. No.: 11/608,466

(22) Filed: Dec. 8, 2006

Related U.S. Application Data
(60) Provisional application No. 60/748,426, filed on Dec. 8, 2005.

(51) Int. Cl. C07K 14/805; C07F 9/02

(52) U.S. Cl. 530/385; 558/155

(57) ABSTRACT

A process for efficiently preparing bis-tetrameric hemoglobin in which the tetramers are specifically linked at predetermined sites on the β-sub-units and in which the tetramers themselves are effectively bonded to prevent dissociation into dimeric αβ-hemoglobin sub-units therefrom is provided. The process uses as cross-linking reagent a hexafunctional aromatic acyl phosphate containing amide groups, The concept is to use a cross-linking reagent which has an excess of site-specific hemoglobin reacting groups, namely six acyl phosphate groups, so that at least four of them will react site specifically to form the bis-tetrameric product of particular therapeutic interest.

### FIELD OF THE INVENTION

This invention relates to hemoglobin, processes and reagents for modifying hemoglobin, and hemoglobin products useful as releasable oxygen carriers in the mammalian body.

### BACKGROUND OF THE INVENTION

Hemoglobin (Hb), which is among the best known proteins, functions as the oxygen delivery system in the circulation of mammals, from the lungs. It is naturally located within the red blood cells (erythrocytes). Hb is well characterized as a tetrameric protein (α₂β₂), of molecular weight 64 kD, with two equivalent αβ dimers, of 32 kD, that are tightly associated with each other, but which are not covalently linked. Outside the erythrocytes, the tetramers reversibly dissociate into αβ dimers,

Extracellular Hb has long been studied and investigated as a potential blood substitute or blood extender, for use in blood transfusions and as an adjunct to whole blood in surgical procedures. Blood typing and matching problems do not present themselves with extracellular Hb, since typing characteristics are associated with erythrocytic cell components such a surface membrane proteins. However, other characteristics such as oxygen carrying capability, oxygen affinity and circulation retention of Hb are notably different in extracellular Hb as compared with intracellular Hb. For example, outside the erythrocytes, purified Hb has a much greater oxygen affinity, and loses the ability to deliver oxygen

Hb αβ dimers are too low in molecular weight to be retained in the circulation system for adequate periods of time. Even extracellular, 64 kD tetrameric Hb has short circulation times and high colloidal osmotic pressure. Efforts have been made to overcome these problems by providing extracellular Hb which is both intermolecularly cross-linked so as to be stabilized in its tetrameric, 64 kD form, and intermolecularly cross-linked to form oligomers (multimers) of tetrameric Hb. Some studies also indicate that materials containing Hbs that are both intermolecularly connected and intramolecularly connected show less of a hypertensive effect than specifically cross-linked Hb tetramers.

Kluger, R.; Lock-O'Brien, J.; and Teytelboym, A., J. Am. Chem Soc. 1999, 121, (29), 6780-6875 reported efficient reagents and methods to produce specifically cross-linked Hb bis-tetramer. Gourianov, N., and Kluger, R., J. Am. Chem. Soc. 2003, 125, (36), 10885-10892, reported several Hb bis-tetramers and their oxygen binding properties.

U.S. Pat. No. 5,811,521 to Kluger and Paai, issued Sep. 22, 1998 describes multifunctional chemical reagents for cross-linking hemoglobin, which are specifically designed to leave at least one of the functionalities free, after reaction with hemoglobin, so that a drug molecule can be bonded to the free functionality, thereby using modified hemoglobin as a drug delivery means. All the reagents specifically disclosed have tribromosalicylate leaving groups. A hexafunctional cross-linking reagent is disclosed, but the results of attempts to cross-link hemoglobin with this reagent are not reported. The reagent is not significantly water soluble, so that it will not be able to react with dissolved Hb.

For in vivo oxygen carrying purposes, it is desirable to provide a cross-linked hemoglobin product which is essentially free of 64 kD intramolecularly cross-linked (or stabilized) Hb tetramer, since this species may be vasoactive. It is also desirable to provide an Hb product in which two or three or more tetrameric Hb units are covalently bonded using specific binding sites on the protein chains. Standardization and control of the binding sites is important in providing a product of predictable and acceptable oxygen binding and releasing characteristics. Some higher molecular weight hemoglobin species have also been reported to be vaso-active, and so it is important that hemoglobin cross-linking processes lead to products where the identity of each cross-linked hemoglobin species produced is known. If a mixture of different cross-linked hemoglobin species is produced, it should be readily separable into its individual components.

A problem addressed by the present invention is the provision of cross-linking reagents that will produce specific multimers of Hb tetramer units in an efficient manner, to the essential exclusion of monomeric Hb tetramer units, and in which the cross-links are formed selectively, as opposed to randomly. A further object is for the production of a product to bind and release oxygen for use as a blood extender in mammalian circulatory systems.

### SUMMARY OF THE INVENTION

From a first aspect the present invention provides polyfunctional cross-linking reagents capable of cross-linking a plurality of hemoglobin units into arrays of molecular weight at least 120 kD, i.e. comprising at least two tetrameric αβαβ or at least four αβ dimeric Hb units. These cross-linking reagents comprise water soluble aromatic amide-phosphate compounds corresponding to the general formula: 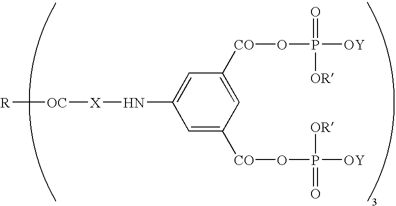
wherein R represents an aromatic nucleus selected from phenyl, naphthyl, phenanthryl, benzanthryl, biphenyl, and binaphthyl;
X represents a direct bond, an amide group, an amino acid residue, a methylene group or a secondary amino group;
R′ represents lower alkyl C₁-C₂; and
Y represents an alkali metal.

Another aspect of the present invention provides a process for preparing multimers of hemoglobin, which comprises reacting hemoglobin in aqueous solution with a water soluble cross-linking reagent as defined above.

The present invention thus provides, from one aspect, a process for efficiently preparing bis-tetrameric hemoglobin in which the tetramers are specifically linked at predetermined sites on the β-sub-units and in which the tetramers themselves are effectively bonded to prevent dissociation into dimeric αβ-hemoglobin sub-units therefrom. The process uses as a cross-linking reagent a hexafunctional aromatic acyl phosphate containing amide groups, as defined above. The concept is to use a cross-linking reagent which has an excess of site-specific hemoglobin reacting groups, namely six acyl phosphate groups, so that at least four of them will react site specifically to form the bis-tetrameric product of particular therapeutic interest. Since two adjacent acyl phosphate groups react with β chains of the hemoglobin tetramer, intramolecular cross-linking is effected also, as well as intermolecular cross-linking. Efficiency of the reaction derives from the selection of amide linkages to provide water solubility so that the reagent and hemoglobin can be reacted together in a common phase, and in the selection of hexafunctional reagents, which ensures that at least four of the groups will react to link tetramers of Hb into stable products of at least 128 kD. More than four of the groups may react with the hemoglobin, to produce products of higher molecular weight, even up to all six of the groups to produce trimers of tetrameric hemoglobin. It is known, however, that it becomes progressively more difficult to bond a second hemoglobin tetramer to a multifunctional cross-linking reagent after a first tetramer has been bonded thereto, and even more difficult to bond a third hemoglobin tetramer after a first and second have bonded thereto. The chances that a quadra-functional cross-linking agent will form a bis-tetramer of hemoglobin, utilizing all four cross-linking groups with 100% efficiency to yield a product containing no significant amounts of unreacted, 64 kD tetrameric hemoglobin, are virtually nil. The chances that four out of six groups of the reagents of the present invention will react, however, is extremely high. Products essentially free of Hb species less than 128 kD are readily obtainable according to the invention.

Specificity of the reaction of the reagents of the present invention derives from the selection of acyl phosphate groups as the reactive groups to react with specific sites of hemoglobin. Epsilon-amine groups are the most accessible and readily reacted with acyl phosphate, and these are found on the β-chains of hemoglobin at position lysine-82 and valine-1, so that this is where reagents of the present invention react. Which of these two sites is actually selected is immaterial.

### BRIEF DESCRIPTION OF THE DRAWINGS

FIG. 1 of the accompanying drawings is the structural chemical formula of a hemoglobin cross-linking reagent according to the present invention;

FIG. 2 is a chemical reaction scheme for synthesizing the reagent illustrated in FIG. 1, and described in the specific experimental examples below;

FIG. 3 shows graphically the results of C4 reverse phase HPLC analysis of the products produced in accordance with the experimental examples below;

FIG. 4 shows graphically the results of size exclusion HPLC analysis of the products produced in accordance with the experimental examples below;

FIG. 5 is a diagrammatic representation of species of cross-linked hemoglobin according to the present invention, and prepared as described in the specific experimental examples below.

### DETAILED DESCRIPTION OF THE INVENTION

The cross-linking reagents in accordance with the invention have six acyl monoalkyl phosphates as the functional, leaving groups, to form amide bonds with the amino residues of Hb. The negative charges on the mono-alkyl phosphate leaving groups make them site-directing groups towards the positively charged protonated amino groups within the DPG-binding site of Hb. Accordingly, the cross-linking reagents of the invention bind specifically to the amino groups on lysine-82 of the β-chains of hemoglobin units, which are disposed in the DPG cleft. The relatively rigid core structure of these cross-linking reagents, resulting from the extended conjugation among the aromatic rings and the amide linkages essentially prevents the compounds from folding onto themselves, so that they maintain a predetermined separation between respective phosphate leaving groups. This separation is designed to be within the appropriate range to cross-link the amino residues both within the same and different Hb tetramers. The separation of amino groups on lysine-82 of the β-chains within a hemoglobin tetrameric unit has been calculated to be approximately 5.1-7.2 Å, and that between the amino groups in two different tetramers of hemoglobin is about 10.6-15.9 Å. Molecular modeling calculations of the cross-linking reagent of the present invention (R=phenyl, R′=methyl in the formula given above) have shown that the distances between the carbonyl groups of the acyl methyl phosphate moieties are within the range to cross-link the amino residues both within the same and different Hb tetramers. This reagent has the potential to cross-link six 32 kD αβ Hb units or three Hb tetramers. Group R in compounds of the present invention are phenyl and naphthyl, with phenyl being more simple and having readily available starting materials for synthesis of the compounds. Methyl as group R′, NH—CO as group X and sodium as group Y, also yield simple compounds and have readily available starting materials for synthesis.

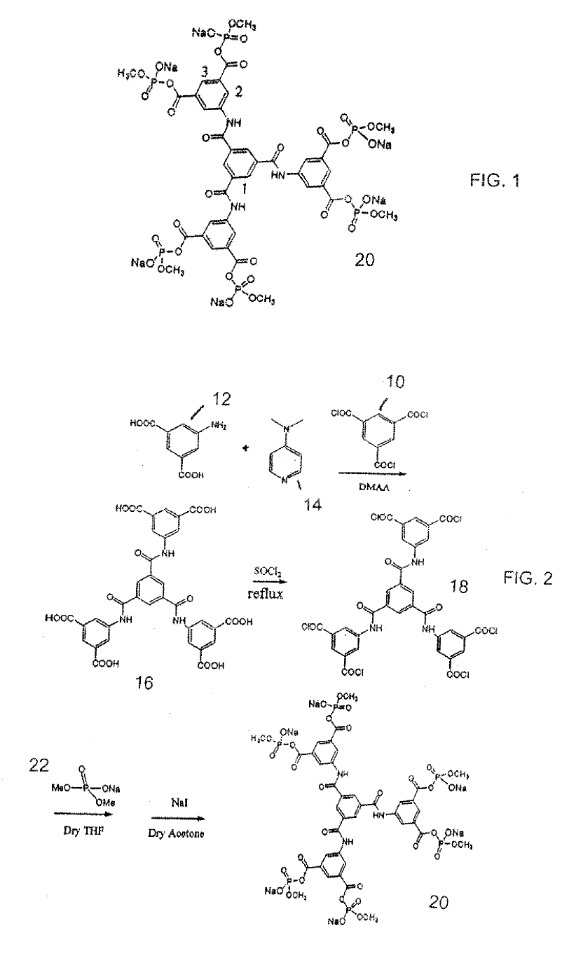

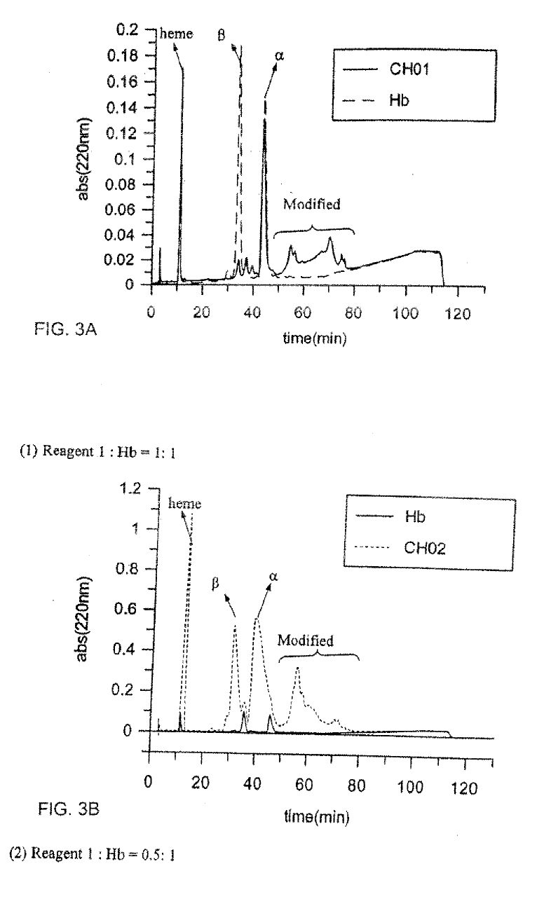

Preparation of the cross-linking reagent of the present invention, depicted in FIG. 1, is diagrammatically illustrated in the reaction scheme presented as FIG. 2 on the accompanying drawings. Commercially available 1,3,5-benzenetricarbonyl chloride 10 is reacted with 5-aminoisophthalic acid 12 in the presence of an HCl scavenger catalyst such as (dimethylamino)-pyridine 14 to remove HCl generated during the reaction, Appropriate stoichiometric relative amounts are used to ensure reaction at all three of the carbonyl chloride sites of compound 10, so as to produce the hexafunctional acid compound 16, This compound is efficiently converted first to the corresponding acyl chloride 18 by reaction with thionyl chloride under reflux, protected under nitrogen, and then to the sodium acyl methyl phosphate of the invention, compound 20, by reaction firstly with sodium methyl phosphate 22 in dry tetrahydrofuran and then with sodium iodide in dry acetone, again protected under nitrogen. The final compound 20 is N,N′,N″-tris[bis(sodium methyl phosphate)isophthalyl]-1,3,5-benzenetricarboxylate. The whole reaction scheme is efficient, and gives yields of compound 20 in excess of 80%. Spectroscopic analysis of the product confirms the illustrated structure of the product 20.

Reaction of cross-linking reagent 20 with hemoglobin is conducted using deoxy Hb and can be carried out in sodium borate buffer solutions under alkaline conditions. Most efficient reactions have been found to take place at about 37° C. at about pH 8.5.

A mixture of products generally results from the reaction of the cross-linking reagent with hemoglobin. To an extent, the composition of the product mixture depends upon the stoichiometric relative amounts of cross-linking reagent and hemoglobin employed in the reaction. If a molar equivalent of cross-linking reagent and at least three molar equivalents of tetrameric hemoglobin is used, the end product will be free of monomeric 64 kD hemoglobin tetramers.

The products and processes of the present invention are further described, for illustrative purposes, in the following specific experimental examples.

### EXAMPLES

### Example 1

### Preparation of Materials

THF was dried by distilling with metallic sodium and acetone was further dried by distilling with mixed Drierite every time before use. Freshly dried THF and acetone were stored under nitrogen. Other commercially available reagents and solvents were applied without further treatment. The reagents used for the HPLC solutions were HPLC grade. Water used to prepare all the buffers and solutions was doubly distilled and deionized. Compounds newly synthesized were characterized by ¹H NMR Spectroscopy, ³¹P NMR Spectroscopy, ESI Mass Spectroscopy and UV-Vis Spectroscopy. ¹H NMR and ³¹P NMR spectra were carried out at 400 MHz and 300 MHz separately. UV-Vis spectroscopy was scanned at room temperature. Molecular modeling studies for the design of the cross-linker molecule were performed using Spartan® '04 for Windows® (Wavefunction, Inc.). Hemoglobin used in this experiment was purified from human whole blood through the method described by Winslow et al¹. Purified hemoglobin was stored in doubly distilled water and stored on ice. Concentrations of the hemoglobin solutions were determined using the cyanomethemoglobin assay described by Tentori and Salvati². The purity of hemoglobin was determined using HPLC analysis developed by Jones³. Modified hemoglobins were characterized by HPLC and SDS-PAGE Gel analysis.

### Example 2

### Synthesis of N,N′,N″-Tris(isophthalyl)-1,3,5-Benzenetricarboxylate, compound 16

5-Aminoisophthalic acid (2.73 g, 15.1 mmol) and 4-(dimethylamino)-pyridine (0.18 g, 1.5 mmol) were dissolved in 50 mL anhydrous N,N-dimethylacetamide under N₂ in a 100 ml, round bottom flask. 1,3,5-Benzenetricarbonyl chloride (1.33 g, 5.0 mmol) was added in. The mixture was stirred under N₂ for 96 hours to give a light yellow solution. The reaction mixture was then transferred to a 250 mL flask. Distilled water (200 mL) was added to precipitate the product as a white fluffy powder. The solid was separated by vacuum filtration and suspended in 150 mL dd water. The solid was then precipitated in the centrifuge set under 10 k RPM for 30 minutes. The supernatant solution was decanted. The solid product was washed 5 times using this process to remove the organic solvent DMAA. The wet solid was lyophilized overnight to give a light yellow crystalline product (3.35 g, 95.8% yield). ¹H NMR(DMSO-d₆). δ 13.38 (broad peak, COOH), 10.96 (s, 3H, CONH), 8.84 (s, 3H, ArH, 1), 8.73(d, 6H, ⁴J=1.2 Hz, ArH, 2), 8.26(t, 3H, ⁴J=1.2 Hz, ArH, 3); MS (ESI, Methanol): C₃₃H₂₁N₃0₁₅ 699.1(M⁻, found), 698.1 ([M-111% 720.1 ([M−2H++Na+]).

### Example 3

### Synthesis of N,N′,N″-Tris[bis(sodium methyl phosphate )isophthalyl]-1,3,5-Benzenetricarboxylate, 20

N,N′,N″-Tris(isophthalyl)-1,3,5-Benzenetricarboxylate (0.28 g, 0.4 mmol) was dissolved in 25 mL thionyl chloride under N₂ and refluxed for 18 hours. Thionyl chloride was then removed by vacuum distillation to give an orange solid. The solid (0.31 g, 0.38 mmol) was dried under vacuum pump for 2 hours to remove thionyl chloride with a trap cooled in liquid nitrogen. Sodium dimethyl phosphate (0.34 g, 2.3 mmol; ¹H NMR (D20): B 3.58 q, ³¹P NMR (D20): 63.0), which was synthesized using trimethyl phosphate and NaI⁴ in dry acetone, was dissolved in 30 mL freshly distilled THF and added into under nitrogen. The mixture was stirred under N₂ for 64 hours to give a yellow solution with some precipitated sodium chloride, which was then removed by vacuum filtration. THF in the filtrate was removed by vacuum distillation. The dark yellow solid obtained was further dried under vacuum pump for 2 hours. Nal (0.346 g, 2.31 mmol) combined with 40 mL dry acetone added under N₂. The mixture was stirred under N₂ for 48 hours. The precipitate was filtered off and washed with dry acetone 5 times. The product (reagent 1, 0.507 g, 90.8% yield) with a slightly yellow color was dried under vacuum pump for 2 hours. ¹H NMR (DMSOd6): 8 11.19 (s, 3H, CONH), 8.91 (s, 3H, ArH, 1), 8.79(d, 6H, ⁴J=1.2 Hz, ArH, 2) 8.25(t, 3H, ⁴J=1.2 Hz, ArH, 3), 3.53(18H, OCH₃); ³¹P-NMR (DMSOd6): 5 6.83; MS (ESI, Dichloromethane): C₃₉H₃₃N₃O₃₃P₆Na₆, doubly charged peaks: 630.5([M⁶⁻+4H⁺]²″12), 583.5([M⁶⁻+4H⁺−94]²12, one phosphate moiety split off), 536.5([M⁶″+4H⁺−2×94]²′12), 489.5([M^{b-}4H⁺−3×94]²ⁿ12), 442.5([M⁶″+4H⁺−4×94]²⁻12), 395.5([M⁶′+4H⁺−5×94]²″12); singly charged peaks: 1263.0([M6″+5H+]′), 1169.0([M⁶″+5H⁺−941⁻), 1075.0([M′⁻+5H−2×94]), 981.0([M−+5H+−3×941−), 887.0([M⁶⁻+5H⁺−4×94]), 793.0([M⁶⁻+5H³⁰ −5×94]⁻). UV absorbance band: 221-225 nm.

The reagent so produced, compound 20, was subjected to UV-Vis spectroscopy. A small amount of reagent 20 was dissolved in doubly distilled water in a quartz cell. The clear solution was scanned from 190 nm to 450 nm using Cintra-® 40 UV-Visible spectrometer. The highest absorbance above 200 nm was at 223 nm.

### Example 4

### Cross-Linking Hb with Reagent 20

Carbonmonoxyhemoglobin (HbCO, 0.5 mL, 0.5 gmol) was passed through a Sephadex G-25 column (250×35 mm) using 0.05 M sodium borate buffer (pH 8.5) at 4° C.

The HbCO buffer solution (−0.1 mM) was oxygenated at 0° C. (ice water pool) and photolyzed under a tungsten lamp for 3 hours to give oxyhemoglobin (OxyHb). OxyHb was then deoxygenated under a stream of N₂ at 37° C. for 3 hours to generate deoxyhemoglobin (deoxyHb).

The first reaction designated CHO1 was conducted at a molar ratio Hb: reagent of 1:1. One equivalent of reagent 20 (0.7 mg, 0.5 μmol) was added into the deoxyHb solution under nitrogen. The reaction was carried out for 18 hours, in some experiments at 20° C. and in others at 37° C., in sodium borate buffer solutions (pH 7.0, pH 8.0, pH 8.5, pH 9.0 and pH 10.0) under N₂. Carbon monoxide was then passed over the modified Hb mixture for 15 minutes to protect the hemes. Modified HbCO was passed through a Sephadex G-25 column (250×35 mm) at 4° C. using 0.1M MOPS buffer (pH 7.2) by ultrafiltration. The mixture was concentrated (membrane size 10 kDa) under 4K RPM for 20 minutes. The concentrated mixture (CHO1, 0.5 mM) was seated and stored at 4° C. A pH of 8.5 and a temperature of 37° C. turned out to be the most efficient.

The second reaction designated reaction CH02 was conducted at a molar ratio Hb:reagent=2:1. 0.7 mg, 0.5 μmol reagent 20 was combined to react with 1.0 mL, 1.0 pmol deoxyHb under the conditions described in reaction CHO1.

The modified Hb solution (CH02) was concentrated and stored at 4° C.

The third reaction designated CH03 used a molar ratio Hb:reagent=1:1.4. 1.0 mg. 0.72 μmol cross-linking reagent 20 was combined to react with 0.5 mL, 0.5 μmol deoxyHb under the conditions described above. The modified Hb solution (CH03) was concentrated and stored at 4° C.

### Example 5

### Isolation of Cross-Linked Hemoglobins

Cross-linked hemoglobin sample (CHO1) was separated by gel filtration chromatography (Sephadex G-100) using 25 mM Tris-HCI, 0.5 M MgCl₂ buffer solution⁴ pH=7.4, to dissociate Hb into dimers. 2 mL of the modified Hb mixture was loaded onto the column (1000×35 mm) and separated by molecular weight. Different fractions with modified Hbs were concentrated by the filter (membrane size 10 kDa) at 4K RPM for 10 minutes. The purity of the separated samples was analyzed by separation through Superdex G-75 size exclusion HPLC and C4 reverse phase analytical HPLC under the conditions described In Example 6 below. Molecular weights of each component were identified by SDS-PAGE Gel analysis, Example 7 below.

### Example 6

### Analysis of Cross-Inked Hemoglobin Performance Liquid Chromatography (HPLC) Analysis

Analytical reverse phase HPLC: The modification of Hb by cross-linking reagent 20 was analyzed by the HPLC analysis procedure developed by Jones³ in 1994. Analytical reverse-phase HPLC with a 330 A pore size C-4 Vydac column (4.6×250 mm) was applied to analyze the globin chain modifications in the reactions CH01, 02 and 03. Gradient elution was applied using 20% acetonitrile (A) and 60% acetonitrile (B) in water with 0.1% (V/V) trifluoroacetic acid. The flow rate was 1 mL/min throughout the analysis. The analytical process for each sample was completed in 120 minutes. The effluent was monitored at 220 nm. The column was equilibrated under the same conditions for analysis before injecting samples. Native Hb (1 μL) was injected to provide a basis for comparison.

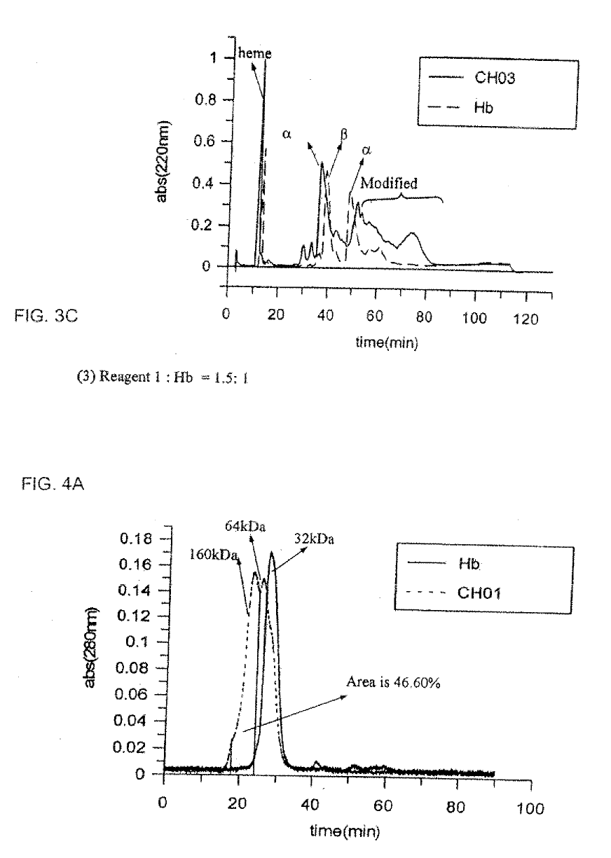

The results are shown graphically on FIG. 3 of the accompanying drawings, Under the running conditions used in the analysis, Hb was dissociated into hemes, α subunits and β subunits, to allow determination of which of the components had been modified by reaction with the cross-linker, in comparison with native hemoglobin. Peaks with different retention times are labeled according to reported values³. The peak for the β subunits was decreased or missing in the products from experiment CHO1 (FIG. 3a) and from experiment CH03 (FIG. 3c) in the modified Hb mixture compared with the native Hb. This showed that the β-subunits are modified in the reaction with reagent 20. The unchanged peaks for heme and α subunits in the modified Hb mixture showed that α subunits remain intact throughout any reaction. These results revealed that the modification occurs selectively through β-subunits.

The relative change of the peak for β subunits indicated how much of the β subunit was modified. In reaction mixtures CHO1 and CH03, FIGS. 3a and 3c respectively, the absence of the peak for the native β subunit suggested that all the β subunit was modified in the reaction with reagent 20. In contrast, a partially decreased peak for the native β subunit in reaction mixture CH02, FIG. 3b, indicates that the modification was incomplete under this combination of reagents. The results from reverse phase HPLC analysis showed that complete modification of Hb could selectively occur on β subunits by sufficient reagent 20.

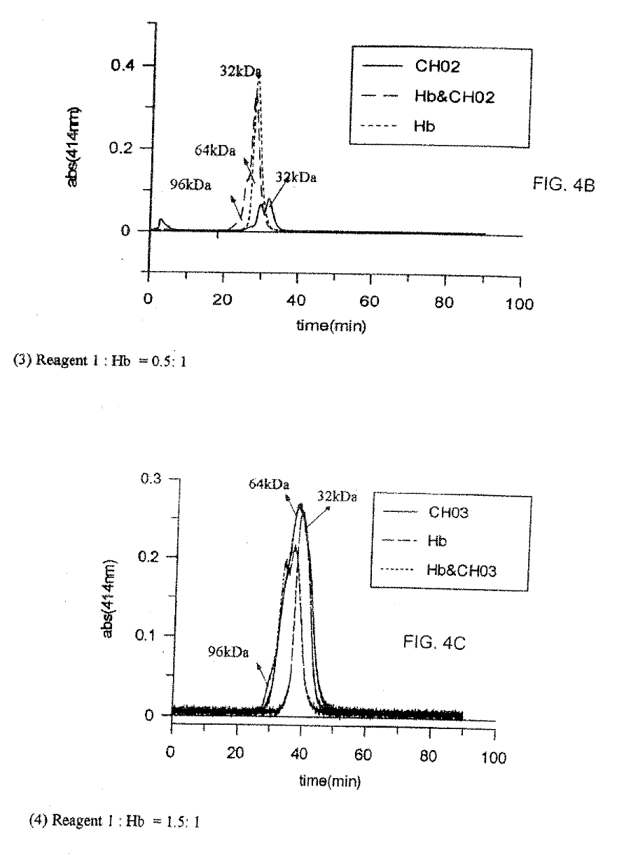

Size exclusion HPLC. Superdex G-75 HR (10×300 mm) was used to investigate the composition of the modified Hb mixture based upon the molecular weights of different components, 20 μL native Hb and 50 ˜100 μL modified sample were eluted separately under conditions to dissociate the Hb tetramer into dimers (Solvent: 25 mM Tris-HCI, 0.5 M MgCl₂ in water, pH 7.4)⁵. 20 μL native Hb and 20 μL modified sample were mixed and eluted under the same conditions to determine the composition of the peaks. The effluent was monitored at 280 nm and 414 nm. The results are shown graphically on accompanying FIG. 4.

Modified Hb samples were detected by their different molecular weights. Hb was eluted under these analytical conditions to give only one peak for αβ dimers (32 kDa). Components coming out before unmodified αβ dimers were cross-linked Hb αβ dimers with higher molecular weights.

In reaction mixture CHO1, FIG. 4a, two peaks coming out before the unmodified αβ dimers are 160 kDa and 64 kDa. Observation of these two peaks is consistent with the results from SDS-PAGE analysis (Example 7 below). Peak area integration showed that the product with a molecular weight of 160 kDa comprised 46.6% of the whole modified Hb mixture CHO1. In contrast, reaction CH03 gave the main product as cross-linked Hb (64 kDa) with two αβ dimmers, FIG. 4c. Reaction CH02, FIG. 4b, contained comparable cross-linked Hb (64 kDa) with two αβ dimers and unmodified αβ dimers (32 kDa). In reaction CH03, FIG. 4c, a cross-linked Hb with a molecular weight of 96 kDa was observed. This product is likely to be cross-linked Hb with three αβ dimers. There were no significant peaks before 64 kDa in CH02 and CH03. Size-exclusion HPLC analysis indicated the best yield of the cross-linked Hb dendrido tetramer with a molecular weight of 160 kDa could be obtained in the reaction with one equivalent each of Hb and reagent 20.

### Example 7

### SDS-PAGE Analysis

Cross-linked Hb mixtures and purified cross-linked Hbs were analyzed by SDS-PAGE under denaturing conditions, so that all non-covalently bonded associates were separated from one another. Protein standards (BTO-RAD, Cat. No. 161-0317; Fermentas, Cat. No. #SM0431), native Hb and modified Hb samples (1-2 μL) were combined with loading buffer to give 20 μL. The 2 times concentrated loading buffer (8 mL) was prepared with 4 mL doubly distilled water, 1.0 mL 0.5 M Tris-HCI, 0.8 ml, glycerol, 1.6 mL 10% SDS, 0.4 mL 2-mercaptoethanol and 0.2 mL 0.5% (w/v) bromophenol blue. The samples were combined with loading buffer and heated at 90° C. for 20 minutes to denature the proteins. 10 μL of each sample was loaded onto a pre-cast polyacrylamide gel (12%, Tris-HCl). The gel was fixed in a mini-PROTEAN® II dual-slab cell apparatus and filled with electrode buffer. The 5 fold concentrated electrode buffer (1 L) consisted of 15 g Tris base, 72 g Glycine and 5 g SDS. The gel was run under a current of 0.04 A.

The gels were stained with staining solution (400 mL methanol, 100 mL glacial acetic acid and 1 g Coomassie blue filled with doubly distilled water to 1 L). Gels soaked in the staining solution were heated in microwave oven for 45 seconds and agitated for 20 minutes. Staining solution was removed and the stained gels were rinsed with destaining solution (400 mL methanol and 100 mL glacial acetic acid filled with doubly distilled water to 1 L) for 3-5 times depending on the stain strength. The destained gels were kept in water over night and scanned using a digital scanner.

In the SDS-PAGE analysis of modified Hb mixtures under different reactant ratios, a control lane containing only native Hb showed a single band for each α and β globin chain, which were estimated to be of an average molecular weight at 16 kDa. The covalently cross-linked subunits remained combined and will show bands at higher molecular weight. Compared with commercial protein samples and the trace amount of undissociated Hb tetramers (64 kD) and αβ dimers (32 kDa) in native Hb sample, the approximate molecular weight of each band can be determined.

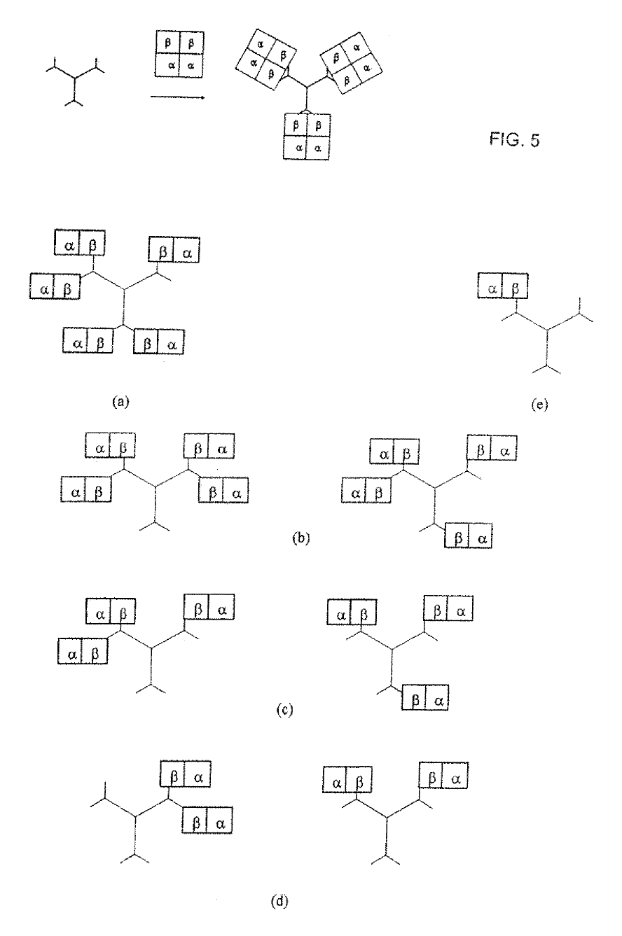

At the top of FIG. 5 is illustrated the general cross-linking reaction and the theoretically possible multimer of three tetrameric Hb units obtainable by the present invention.

Two main bands appeared With higher molecular weights than the α and β subunits (16 kDa) in reaction mixture CHOI. One band possessing a molecular weight of 80 kDa results from five β subunits linked together, i.e. corresponding to product 5a on FIG. 5 but with its α units dissociated away due to the denaturing conditions. The other band, at 32 kDa, would be Hb cross-linked between two β subunits, FIG. 5d. In reaction mixture CHO1, there were also minor bands at around 64 kDa (four β subunits linked together as a Hb bis-tetramers, products 5b) and 48 kDa (three β subunits linked together, products 5c). In contrast, within reaction mixture CH02 and CH03, most cross-linked Hb product was Hb cross-linked between two β subunits (32 kDa, products 5d), There was no significant band observed with higher molecular weight than 32 kDa in reaction mixture CH02, while a band appeared at 48 kDa in reaction mixture CH03. Comparing the results of size-exclusion HPLC analysis, there could be determined the relative composition of modified Hb species in the reaction mixture.

Single peaks observed in both size-exclusion HPLC and reverse phase HPLC showed the purity of the Hb dendrimer (160 kDa) sample isolated from the reaction mixture, SDS-PAGE analysis gave the molecular weight of each component. Trace amounts at 32 kDa were from αβ dimers from the incomplete cleavage of disulfides.

### Example 8

### Molecular Modeling

Molecular modeling of reagent 20 was carried out using Spartan Semi-empirical calculations by minimizing the molecular energy. The conformation of the whole molecule is like a fan with three leaves. The calculation showed that the distances between the carbonyl carbon centers in the acyl phosphate moieties are about 12 Å (on different benzene rings) and 5 Å (on the same benzene ring).

Molecular modeling calculations showed the distances between acyl phosphate groups of reagent 20 are in the scale to both intra- and inter-molecularly crosslink Hbs. However, the span of the molecule may still influence the cross-linking of three hemoglobin tetramers as one multimer. As a relatively large protein molecule (64×55×50 Å), the third Hb tetramer has to overcome a significant steric hindrance caused by pre-cross-linked Hb with four αβ dimers so as to react with the linker molecule. It is reasonable that only one of the two αβ domains in the third Hb could get the chance to form the amide bond with the rest two available methyl phosphate functional groups of the linker molecule before the hydrolysis. This could also account for the production of this multimeric Hb with five αβ dimers.

Multimers of hemoglobin according to the present invention are potentially useful as oxygen carriers for mammalian patients, as substitutes for red blood cells and as blood extenders. They are also potentially useful in other medical and biochemical areas where hemoglobin-based inter-molecularly cross-linked and intra-molecularly products have previously been proposed for use, for example as oxygen-delivering therapeutic adjuncts for radiation and chemotherapy for cancer patients, as oxygen-delivery therapeutics for treating ischemic conditions, as diagnostic reagents to provide contrast media for MRI and PET scanning diagnoses, and as components of cell culture medium otherwise calling for whole blood as a medium component. Other potential related applications will be apparent to those of skill in the art.

### REFERENCES

1. Vandegriff, K. D.; Malavalli, A; Wooldridge, J; Lohman, I; Winslow, R. M; Transfusion 2003, 43,(4), 509-516.
2. Tentori, L.; Salvati, A. M., Hemoglobin, Pt B 1981, 76, 705-715.
3. Jones, R. T., Hemoglobin, Pt. B 1994, 231, 322-343.
Zervas, L.; Dilaris I., J Am Chem Soc 1955, 77, (20), 5354-5357.
Guidotti, G., J Biol Chem 1967, 242, (16), 3685-&

What is claimed is:

1. Polyfunctional cross-linking reagents capable of cross-linking a plurality of hemoglobin units into multimers of molecular weight at least 120 kD, comprising water soluble aromatic amide-phosphate compounds correspondlng to the general formula: 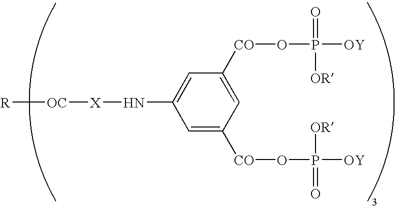
wherein R represents an aromatic nucleus selected from phenyl, naphthyl, phenanthryl, benzanthryt, biphenyland binaphthyl;
R′ represents lower alkyl C₁-C₂;
X represents a direct bond, a methylene group, an amide group, a secondary amine group or an amino acid residue; and
Y represents an alkali metal.

2. Reagents according to claim 1, wherein R represents phenyl.

3. Reagents according to claim 2, wherein the phenyl group is symmetrically substituted with the three amido-phenyl diphosphate groups.

4. Reagents according to claim 3, wherein R′ represents methyl.

5. Reagents according to claim 4, wherein X represents sodium.

6. A reagent according to claim 1 which is N,N′,N″-tris[bis(sodium methyl phosphate)isophthalyl]-1,3,5-benzenetricarboxylate, of formula:

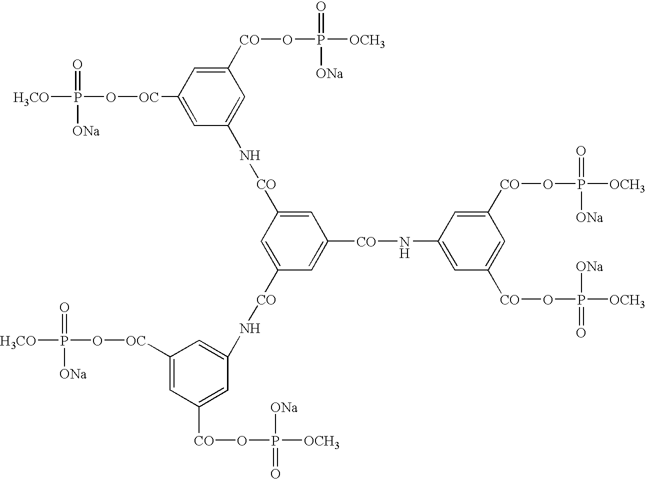

7. An inter-molecularly cross-linked hemoglobin product comprising at least three dimeric 32 kD αβ Hb units, the β sub-units of each unit being covalently bonded through an amide linkage involving lysine-82 or valine-1 of the protein chain, directly or indirectly, to a common aromatic nucleus residue of a cross-linking reagent, the product being essentially free of hemoglobin species of 64 kD and less.

8. The product according to claim 7 consisting essentially of a mixture of hemoglobin species having two tetrameric Hb units, and hemoglobin species having three cross-linked tetrameric Hb units.

9. The product according to claim 7, wherein the nucleus residue of the cross-linking reagent is N,N′,N″-tris(isophthalyl)-1,3,5-benzene tricarboxylate, of formula:

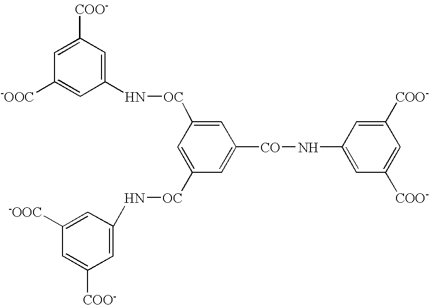

10. The product according to claim 9, wherein the residue derives from the cross-linking reagent is N,N′,N″-tris[bis(sodium methyl phosphate)isophthalyl]-1,3,5-benzenetricarboxylate.

11. A process of preparing multimers of hemoglobin which comprises reacting hemoglobin in aqueous solution with a hexafunctional cross-linking reagent as defined in any of claims 1-6.

12. A process of delivering oxygen to tissues and organs of a mammalian patient, which comprises administering to the blood stream of a mammalian patient in need thereof an effective amount an inter-molecularly cross-linked hemoglobin product as defined any of claims 1-10 in admixture with a pharmaceutically acceptable carrier.

13. Use in the preparation or manufacture of an oxygen carrier for administration to a mammalian patient for delivery of oxygen to tissues and organs of the patient, of an inter-molecularly cross-linked hemoglobin product as defined any of claims 1-10.

14. Use in the preparation or manufacture of diagnostic reagents to provide contrast media for MRI and PET scanning diagnoses, of an inter-molecularly cross-linked hemoglobin product as defined any of claims 1-10.

15. A cell culture medium including an effective amount an inter-molecularly cross-linked hemoglobin product as defined any of claims 1-10.
